(19) United States
(12) Patent Application Publication
(10) Pub. No.: US 2007/0098183 A1
(43) Pub. Date: May 3, 2007

(54) Acoustic signal reproduction apparatus

(75) Inventors: Akihiko Enamito, Kawasaki-shi (JP); Takahiro Hiruma, Fuchu-shi (JP)

Correspondence Address: Charles N.J. Ruggiero;Ohlandt, Greeley, Ruggiero & Perle, L.L.P., 10th Floor, One Landmark Square, Stamford, CT, 06901-2682, US

(73) Assignee: KABUSHIKI KAISHA TOSHIBA

(21) Appl. No.: 11/586,236

(22) Filed: Oct. 25, 2006

(30) Foreign Application Priority Data
Oct. 25, 2005 (JP) 2005-310114

(51) Int. Cl. A61F 11/06; G10L 21/00

(52) U.S. Cl. 381/71.1; 704/278

(57) ABSTRACT

A reproduction apparatus includes an acoustic signal generator which generates an acoustic signal, first sound pressure detection points located at N points in an audible area to detect sound pressure signals, N+1 control sound wave generators each of which emits a sound wave based on the acoustic signal to generate a control sound, second sound pressure detection points located at M points in a non-audible area to detect sound pressure signals, M sound wave generators each of which emits a sound wave based on the acoustic signal to generate a main sound, and a controller to control an amplitude and a phase of each of the (N+1) control sound wave generators.

### CROSS-REFERENCE TO RELATED APPLICATIONS

This application is based upon and claims the benefit of priority from prior Japanese Patent Application No. 2005-310114, filed Oct. 25, 2005, the entire contents of which are incorporated herein by reference.

### BACKGROUND OF THE INVENTION

1. Field of the Invention

The present invention relates to an acoustic signal reproduction apparatus that separates a sound field into an audible (non sound reduction) area and a non-audible (sound reduction) area.

2. Description of the Related Art

JP-A No. 2001-112083 (KOKAI) describes a semi dispersion loudspeaking scheme in which a main sound source speaker (main speaker) is located in the vicinity of a sound source and a dispersion speaker is located on a ceiling away from the sound source. This document describes control performed so that the sounding time of the main speaker is delayed behind that of the dispersion speaker so as to give, even at a sound reception point close to the dispersion speaker, the feeling that the sound comes from a speaker direction (sound image localization in a direction that matches the speaker direction).

With an audible area separation method, a main sound source and a control sound source which have different range attenuations are located in an integral structure in proximity to each other. Then, a sound (contents sound reproduced by the main sound source) is not reduced in the vicinity of these sound sources but is reduced at a long distance from them to separate the sound field into an audible area and a non-audible area. Even if sound waves from these two sound sources are emitted to the space so as to travel in proximity to each other, in a low sound range with a large wavelength (low frequency area), sound reduction may occur in the vicinity of the sound source, that is, in the audible area. This disadvantageously prevents a proper sound field resolution from being obtained.

### BRIEF SUMMARY OF THE INVENTION

An acoustic signal reproduction apparatus according to an aspect of the present invention comprises an acoustic signal generator which generates an acoustic signal, first sound pressure detection points located at N (N is a natural number) points in an audible area to detect sound pressure signals, N+1 control sound wave generators each of which emits a sound wave based on the acoustic signal to generate a control sound, second sound pressure detection points located at M (M is a natural number) points in a non-audible area to detect sound pressure signals, M sound wave generators each of which emits a sound wave based on the acoustic signal to generate a main sound, and a controller configured to control an amplitude and a phase of each of the (N+1) control sound wave generators so as to suppress a sum of first sound pressure signals based on control sounds generated by the N+1 control sound wave generators, the first sound pressure signals being detected by the N first sound pressure detection points, and to suppress a sum of second sound pressure signals from the N+1 sound wave generators and second sound pressure signals from the M sound wave generators, the second pressure signals being detected by the M sound pressure detection points.

### BRIEF DESCRIPTION OF THE SEVERAL VIEWS OF THE DRAWING

FIG. 1 is a block diagram showing an acoustic signal reproduction apparatus according to an embodiment;

FIGS. 2A to 2C are diagrams showing a procedure of identifying a space transfer function to calculate a control filter;

FIG. 3 is a diagram showing the appearance of a 6×8 integral speaker;

FIG. 4 is a functional block diagram showing transfer function identification means;

FIG. 5 is a functional block diagram showing control filter calculation means;

FIG. 6 is a diagram showing various examples of configurations comprising sound wave generation sections (speakers) having differently shaped sound wave generation surfaces;

FIG. 7 is a graph showing the relationship between the distance [m] from a sound source and sound pressure (difference);

FIGS. 8A and 8B are diagrams showing a difference in distance resulting from a difference in the shape of sound wave generation surface of the sound wave generation section (speaker);

FIGS. 9A to 9D are model diagrams showing that the present invention is applied to a television;

FIGS. 10A to 10C are diagrams showing the results of control under which a control sound source group in the 6×8 integral speaker (FIG. 3) is divided into two; and

FIGS. 11A to 11C are diagrams showing the results of control under which the control sound source group in the 6×8 integral speaker (FIG. 3) is divided into three.

### DETAILED DESCRIPTION OF THE INVENTION

### First Embodiment

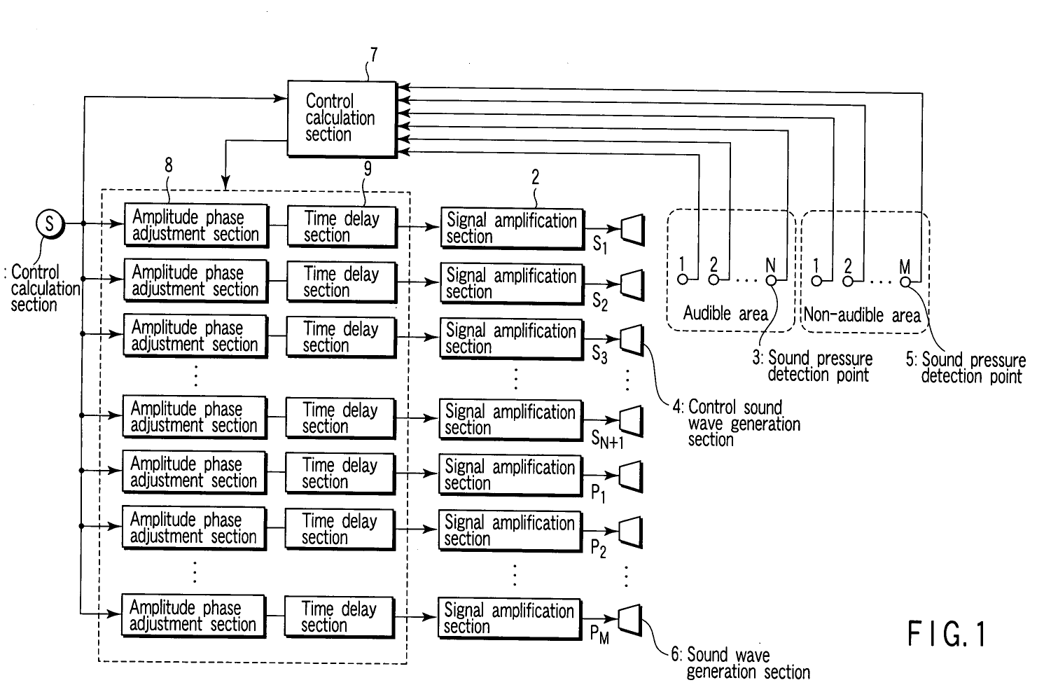

Referring to FIG. 1, an acoustic signal reproduction apparatus according to a first embodiment comprises an acoustic signal generation section 1, signal amplification sections 2, sound pressure detection points 3, control sound wave generation sections 4, M sound pressure detection points 5, M sound wave generation sections 6, a control calculation section 7, amplitude phase adjustment sections 8, and time delay sections 9.

The sound pressure detection points 3 are, for example, microphones that detect sound pressure. The N (N is a natural number) sound pressure detection points 3 are arranged at predetermined positions in an audible area (non sound reduction area).

Each of the control sound wave generation sections 4 is a speaker (control sound source) that emits the sound wave of a control sound. The N+1 control sound wave generation sections 4 are arranged in the apparatus. As described below, each of the control sound sources comprises a point sound source, a line sound source, or a surface sound source. The control sound is generated as described below. The acoustic signal generation section 1 generates an acoustic signal, which is processed by the amplitude phase adjustment section 8 and time delay section 9. The resulting signal is then amplified by signal amplification section 2.

The sound pressure detection points 5 are, for example, microphones that detect sound pressure. The M (M is a natural number) sound pressure detection points 5 are arranged at predetermined positions in a non-audible area (sound reduction area).

Each of the sound wave generation sections 6 is a speaker (main sound source) that emits the sound wave of a contents sound (main sound). The M sound wave generation sections 6 are arranged in the apparatus.

As described below, each of the control sound sources comprises a point sound source, a line sound source, or a surface sound source. The contents sound is generated as described below. The acoustic signal generation section 1 generates an acoustic signal, which is processed by the amplitude phase adjustment section 8 and time delay section 9. The resulting signal is then amplified by signal amplification section 2.

The control calculation section 7 controls each of the control sound sources comprising a plurality of speakers. The control calculation section 7 calculates an amplitude, a phase, and a delay time such that the sum of sound pressure signals from the N+1 control sound wave generation sections 4 is suppressed, preferably minimized, at each of the N sound pressure detection points 3 and such that the sum of sound pressure signals from the N+1 control sound wave generation sections 4 and sound pressure signals from the M sound pressure generation sections 6 is suppressed, preferably minimized, at each of the M sound pressure detection points 5.

With sound field area separation, an audible area (non sound reduction area) is formed around the N sound pressure detection points 3, while a non-audible area (sound reduction area) is formed around the M sound pressure detection points 5. The sound field area separation is achieved by using differences in range attenuation factor among the sound sources to allow sound waves with different range attenuation factors to interfere with one another. In this case, the amplitude and phase are calculated on the basis of a volume to velocity ratio.

The following is based on a determinant described below: the relationship among the number N of the arranged voltage detection points 3, the number N+1 of the arranged control sound wave generation sections 4, the number M of the arranged sound pressure detection points 5, and the number M of the arranged sound wave generation sections 6. At least two sound wave generation sections 4 are arranged in the apparatus. At least one sound pressure detection point 5 needs to be placed in the apparatus.

Now, description will be given below of basic concept of control, by the control calculation section 7, of the control sound sources comprising the plurality of speakers.

[Audible Area]

A plurality of evaluation points are set in the audible area. The sound pressure synthesis value for control sound source groups on each of the evaluation points is adjusted to zero. Here, the number of control sound source groups is the number of the evaluation points plus 1. In the description below, N evaluation points are set, that is, N+1 (qₛ₁, qₛ₂, . . . , q_{sN+1}) control sound source groups are arranged.

The evaluation points in the audible area are denoted by n₁, n₂, . . . , n_{N}. The synthetic sound pressures are denoted by Pn₁, Pn₂, . . . Pn_{N}.
Pₙ₁=Fₛ₁ₙ₁·qₛ₁+Fₛ₂ₙ₁·qₛ₂+ . . . +F_{s(N+1)n1}·q_{s(N+1)}
Pₙ₂=Fₛ₁ₙ₂·qₛ₁+Fₛ₂ₙ₂·qₛ₂+ . . . +F_{s(N+1)n2}·q_{s(N+1)}
P_{nN}=F_{s1nN}·qₛ₁+F_{s2nN}·qₛ₂+ . . . +F_{s(N+1)nN}·q_{s(N+1)}

Thus, the sound pressure is determined from a complex amplitude qₛ multiplied by a space transfer function F. The synthetic sound pressure P at each evaluation point can be expressed by:

$$\begin{matrix}P = [ \begin{matrix}\begin{matrix}\begin{matrix}{P}_{n 1} \\ {P}_{n 2}\end{matrix} \\ ⋮\end{matrix} \\ {P}_{n 2}\end{matrix} ] = [ \begin{matrix}{F}_{s 1 n 1} & {F}_{s 2 n 1} & ⋯ & {F}_{s ( N + 1 ) n 1} \\ {F}_{s 1 n 2} & ⋰ & & ⋮ \\ ⋮ & & ⋰ & ⋮ \\ {F}_{s 1 nN} & ⋯ & ⋯ & {F}_{s ( N + 1 ) nN}\end{matrix} ] \cdot [ \begin{matrix}\begin{matrix}\begin{matrix}{q}_{s 1} \\ {q}_{s 2}\end{matrix} \\ ⋮\end{matrix} \\ {q}_{s ( N + 1 )}\end{matrix} ] \\ = [ \begin{matrix}{F}_{s 2 n 1} & {F}_{s 3 n 1} & ⋯ & {F}_{s ( N + 1 ) n 1} \\ {F}_{s 2 n 2} & ⋰ & & ⋮ \\ ⋮ & & ⋰ & ⋮ \\ {F}_{s 2 nN} & ⋯ & ⋯ & {F}_{s ( N + 1 ) nN}\end{matrix} ] \cdot [ \begin{matrix}\begin{matrix}\begin{matrix}{q}_{s 2} \\ {q}_{s 3}\end{matrix} \\ ⋮\end{matrix} \\ {q}_{s ( N + 1 )}\end{matrix} ] + [ \begin{matrix}\begin{matrix}\begin{matrix}{F}_{s 1 n 1} \\ {F}_{s 1 n 2}\end{matrix} \\ ⋮\end{matrix} \\ {F}_{s 1 nN}\end{matrix} ] \cdot {q}_{s 1}\end{matrix}$$

When the sound pressure synthesis is zero, that is, P [symbol] 0, the second and subsequent complex amplitudes can be expressed by Equation (1) using the first complex amplitude and space transfer function and their inverse matrix.

$$[ \begin{matrix}\begin{matrix}\begin{matrix}{q}_{s 2} \\ {q}_{s 3}\end{matrix} \\ ⋮\end{matrix} \\ {q}_{s ( N + 1 )}\end{matrix} ] = - {[ \begin{matrix}{F}_{s 2 n 1} & {F}_{s 3 n 1} & ⋯ & {F}_{s ( N + 1 ) n 1} \\ {F}_{s 2 n 2} & ⋰ & & ⋮ \\ ⋮ & & ⋰ & ⋮ \\ {F}_{s 2 nN} & ⋯ & ⋯ & {F}_{s ( N + 1 ) nN}\end{matrix} ]}^{- 1} \cdot [ \begin{matrix}\begin{matrix}\begin{matrix}{F}_{s 1 n 1} \\ {F}_{s 1 n 2}\end{matrix} \\ ⋮\end{matrix} \\ {F}_{s 1 nN}\end{matrix} ] \cdot {q}_{s 1} \qquad ( 1 )$$

Here, the following equation is given.

$${F}_{s}^{″} = [ \begin{matrix}{F}_{s 2 n 1} & {F}_{s 3 n 1} & ⋯ & {F}_{s ( N + 1 ) n 1} \\ {F}_{s 2 n 2} & ⋰ & & ⋮ \\ ⋮ & & ⋰ & ⋮ \\ {F}_{s 2 nN} & ⋯ & ⋯ & {F}_{s ( N + 1 ) nN}\end{matrix} ]$$

The complex amplitude of the control sound source group is expressed by:

$$[ \begin{matrix}\begin{matrix}\begin{matrix}{q}_{s 2} \\ {q}_{s 3}\end{matrix} \\ ⋮\end{matrix} \\ {q}_{s ( N + 1 )}\end{matrix} ] = - {F}_{s}^{″ - 1} \cdot [ \begin{matrix}\begin{matrix}\begin{matrix}{F}_{s 1 n 1} \\ {F}_{s 1 n 2}\end{matrix} \\ ⋮\end{matrix} \\ {F}_{s 1 nN}\end{matrix} ] \cdot {q}_{s 1}$$

[Non-Audible Area]

A plurality of evaluation points are set in the non-audible area. The synthetic sound pressures from main sound source groups and control sound source groups on each of the evaluation points are adjusted to zero. Here, the number of main sound source groups is the same as that of control sound source groups. In the description below, M evaluation points are set, that is, M (qₚ₁, qₚ₂, . . . , q_{pM}) main sound source groups are arranged. The main sound source qₚ is handled as a reference signal (that is, a simple constant).

The evaluation points in the non-audible area are denoted by m₁, m₂, . . . , m_{N}. The synthetic sound pressures are denoted by Qm₁, Qm₂, . . . Qm_{M}.
Qₘ₁=Zₚ₁ₘ₁·qₚ₁+Zₚ₂ₘ₁·qₚ₂+ . . . +Z_{pMm1}·q_{pM}+Zₛ₁ₘ₁·qₛ₁+Zₛ₂ₘ₁·qₛ₂+ . . . +Z_{s(N+1)m1}·q_{s(N+1)}
Qₘ₂=Zₚ₁ₘ₂·qₚ₁+Zₚ₂ₘ₂·qₚ₂+ . . . +Z_{pMm2}·q_{pM}+Zₛ₁ₘ₂·qₛ₁+Zₛ₂ₘ₂·qₛ₂+ . . . +Z_{s(N+1)m2}·q_{s(N+1)}
Q_{mM}=Z_{p1mM}·qₚ₁+Z_{p2mM}·qₚ₂+ . . . +Z_{pMmM}·q_{pM}+Z_{s1mM}·qₛ₁+Z_{s2mM}·qₛ₂+ . . . +Z_{s(N+1)mM}·q_{s(N+1)}

Each synthetic sound pressure Q is expressed by:

$$Q = [ \begin{matrix}\begin{matrix}{Q}_{m 1} \\ {Q}_{m 2}\end{matrix} \\ \begin{matrix}⋮ \\ {Q}_{mM}\end{matrix}\end{matrix} ] = [ \begin{matrix}{Z}_{p 1 m 1} & {Z}_{p 2 m 1} & ⋯ & {Z}_{pMm 1} \\ {Z}_{p 1 m 2} & ⋰ & & ⋮ \\ ⋮ & & ⋰ & ⋮ \\ {Z}_{p 1 mM} & ⋯ & ⋯ & {Z}_{pMmM}\end{matrix} ] \cdot [ \begin{matrix}\begin{matrix}\begin{matrix}{q}_{p 1} \\ {q}_{p 2}\end{matrix} \\ ⋮\end{matrix} \\ {q}_{pM}\end{matrix} ] + \\ [ \begin{matrix}\begin{matrix}\begin{matrix}{Z}_{s 1 m 1} \\ {Z}_{s 1 m 2}\end{matrix} \\ ⋮\end{matrix} \\ {Z}_{s 1 mM}\end{matrix} ] \cdot {q}_{s 1} + [ \begin{matrix}{Z}_{s 2 m 1} & {Z}_{s 3 m 1} & ⋯ & {Z}_{s ( N + 1 ) m 1} \\ {Z}_{s 2 m 2} & ⋰ & & ⋮ \\ ⋮ & & ⋰ & ⋮ \\ {Z}_{s 2 mM} & ⋯ & ⋯ & {Z}_{s ( N + 1 ) mM}\end{matrix} ] \cdot [ \begin{matrix}\begin{matrix}\begin{matrix}{q}_{s 2} \\ {q}_{s 3}\end{matrix} \\ ⋮\end{matrix} \\ {q}_{s ( N + 1 )}\end{matrix} ]$$

Here, the following equation is given.

$${Z}_{p} = [ \begin{matrix}{Z}_{p 1 m 1} & {Z}_{p 2 m 1} & ⋯ & {Z}_{pMm 1} \\ {Z}_{p 1 m 2} & ⋰ & & ⋮ \\ ⋮ & & ⋰ & ⋮ \\ {Z}_{p 1 mM} & ⋯ & ⋯ & {Z}_{pMmM}\end{matrix} ]$$

Then, the following equation is given.

$$\begin{matrix}Q = {Z}_{p} \cdot [ \begin{matrix}\begin{matrix}\begin{matrix}{q}_{p 1} \\ {q}_{p 2}\end{matrix} \\ ⋮\end{matrix} \\ {q}_{pM}\end{matrix} ] + [ \begin{matrix}\begin{matrix}\begin{matrix}{Z}_{s 1 m 1} \\ {Z}_{s 1 m 2}\end{matrix} \\ ⋮\end{matrix} \\ {Z}_{s 1 mM}\end{matrix} ] \cdot {q}_{s 1} + [ \begin{matrix}{Z}_{s 2 m 1} & {Z}_{s 3 m 1} & ⋯ & {Z}_{s ( N + 1 ) m 1} \\ {Z}_{s 2 m 2} & ⋰ & & ⋮ \\ ⋮ & & ⋰ & ⋮ \\ {Z}_{s 2 mM} & ⋯ & ⋯ & {Z}_{s ( N + 1 ) mM}\end{matrix} ] \cdot \\ [ - {F}_{s}^{″ - 1} \cdot [ \begin{matrix}\begin{matrix}\begin{matrix}{F}_{s 1 n 1} \\ {F}_{s 1 n 2}\end{matrix} \\ ⋮\end{matrix} \\ {F}_{s 1 nN}\end{matrix} ] \cdot {q}_{s 1} ] \\ = {Z}_{p} \cdot [ \begin{matrix}\begin{matrix}\begin{matrix}{q}_{p 1} \\ {q}_{p 2}\end{matrix} \\ ⋮\end{matrix} \\ {q}_{pM}\end{matrix} ] + [ [ \begin{matrix}\begin{matrix}\begin{matrix}{Z}_{s 1 m 1} \\ {Z}_{s 1 m 2}\end{matrix} \\ ⋮\end{matrix} \\ {Z}_{s 1 mM}\end{matrix} ] - [ \begin{matrix}{Z}_{s 2 m 1} & {Z}_{s 3 m 1} & ⋯ & {Z}_{s ( N + 1 ) m 1} \\ {Z}_{s 2 m 2} & ⋰ & & ⋮ \\ ⋮ & & ⋰ & ⋮ \\ {Z}_{s 2 mM} & ⋯ & ⋯ & {Z}_{s ( N + 1 ) mM}\end{matrix} ] \cdot \\ {F}_{s}^{″ - 1} \cdot [ \begin{matrix}\begin{matrix}\begin{matrix}{F}_{s 1 n 1} \\ {F}_{s 1 n 2}\end{matrix} \\ ⋮\end{matrix} \\ {F}_{s 1 nN}\end{matrix} ] ] \cdot {q}_{s 1}\end{matrix}$$

When the sound pressure synthesis value is zero, that is, Q [symbol] 0, the complex amplitude of the first control sound source can be expressed by:

$${q}_{s 1} = {[ [ \begin{matrix}\begin{matrix}\begin{matrix}{Z}_{s 1 m 1} \\ {Z}_{s 1 m 2}\end{matrix} \\ ⋮\end{matrix} \\ {Z}_{s 1 mM}\end{matrix} ] - [ \begin{matrix}{Z}_{s 2 m 1} & {Z}_{s 3 m 1} & ⋯ & {Z}_{s ( N + 1 ) m 1} \\ {Z}_{s 2 m 2} & ⋰ & & ⋮ \\ ⋮ & & ⋰ & ⋮ \\ {Z}_{s 2 mM} & ⋯ & ⋯ & {Z}_{s ( N + 1 ) mM}\end{matrix} ] \cdot {F}_{s}^{″ - 1} \cdot [ \begin{matrix}\begin{matrix}\begin{matrix}{F}_{s 1 n 1} \\ {F}_{s 1 n 2}\end{matrix} \\ ⋮\end{matrix} \\ {F}_{s 1 nN}\end{matrix} ] ]}^{- 1} {Z}_{p} \cdot [ \begin{matrix}\begin{matrix}\begin{matrix}{q}_{p 1} \\ {q}_{p 2}\end{matrix} \\ ⋮\end{matrix} \\ {q}_{pM}\end{matrix} ] \qquad ( 2 )$$

Substituting Equation (2) into Equation (1) enables the calculation of qₛ₂, qₛ₃, . . . , q_{sN+1}.

On the basis of the above basic concept, the control calculation section 7 can control each of the control sound sources comprising the plurality of speakers and arranged in proximity to (integrated with) the main sound source. For audible area separation utilizing the differences in range attenuation factor among the sound sources, the control calculation section 7 performs control by calculating the amplitude, phase, and delay time such that the sum of sound pressure signals from the N+1 control sound wave generation sections 4 is suppressed at each of the N sound pressure detection points 3 and such that the sum of sound pressure signals from the N+1 control sound wave generation sections 4 and sound pressure signals from the M sound pressure generation sections 6 is suppressed at each of the M sound pressure detection points 5, as described above. This enables appropriate audible area separation to be achieved even in a low sound range. That is, low frequency sounds have relatively large wavelengths, and the main sound source is located in proximity to the control sound source. This prevents a propagation path difference from being obtained. This problem can be solved by precisely controlling the control sound source.

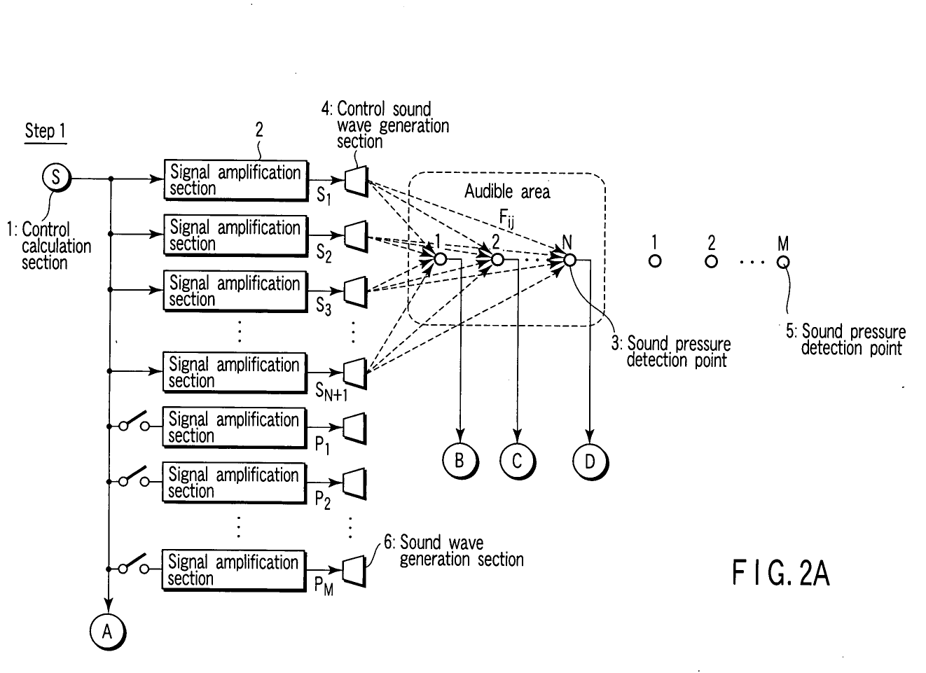

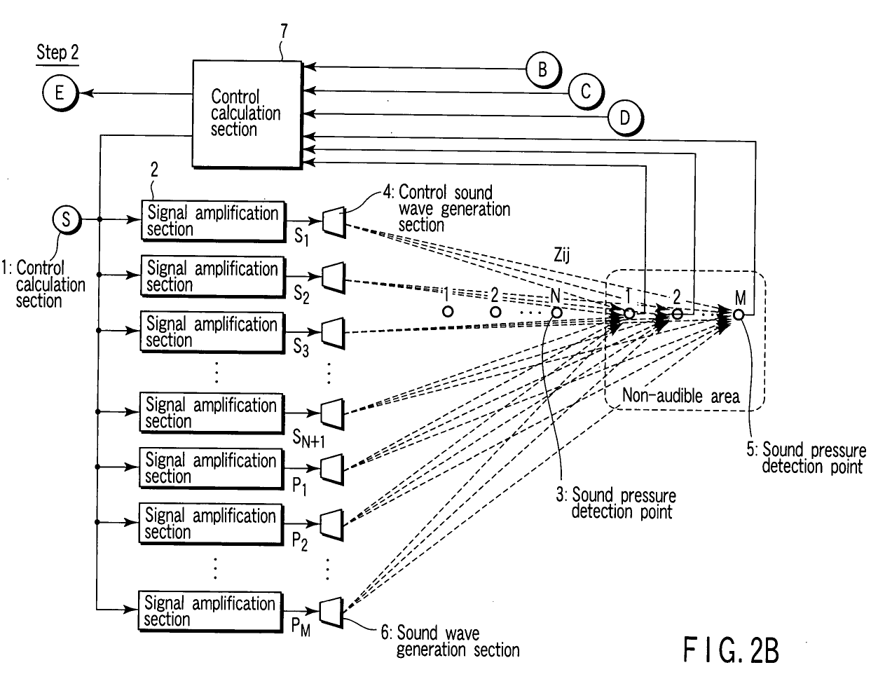

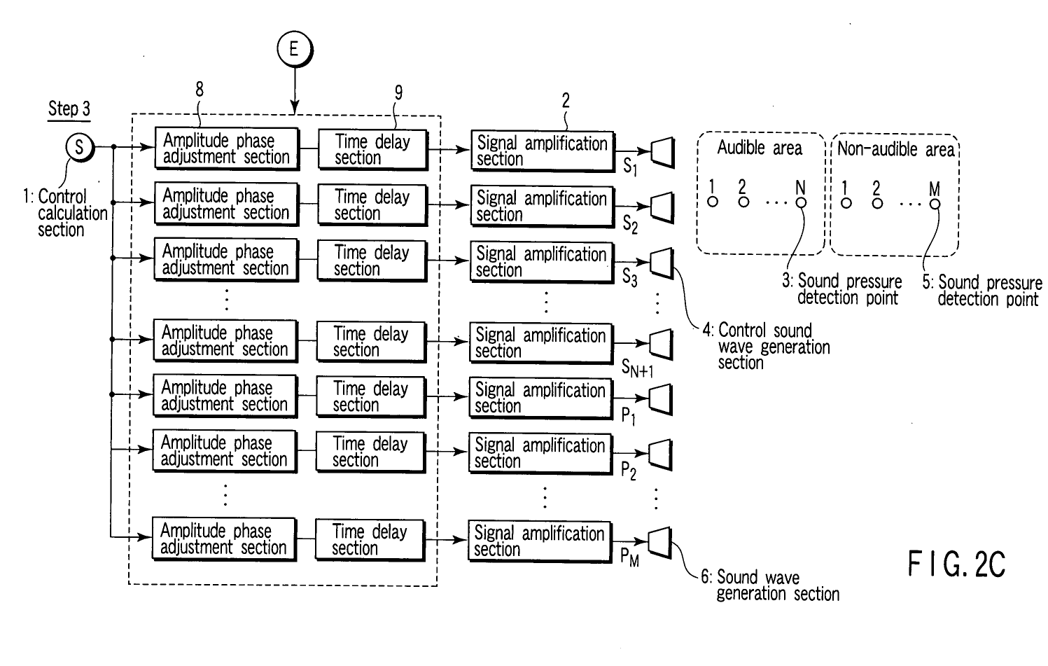

Implementation of an acoustic signal reproduction apparatus requires identification of required space transfer functions and calculation of a control filter. Specifically, as shown in FIG. 2, in step 1, a space transfer function Fij for the audible area is identified. In step 2, a space transfer function Zij for the non-audible area is identified, and the control calculation section 7 then calculates a control filter. The control calculation section 7 provides the calculated control filter to the amplitude phase adjustment section 8 and time delay section 9. The identification of the required space transfer functions and the calculation of the control filter will be described in a second embodiment and a third embodiment, respectively. In step 3, audible sound separation is carried out to reproduce the contents sound.

### Second Embodiment

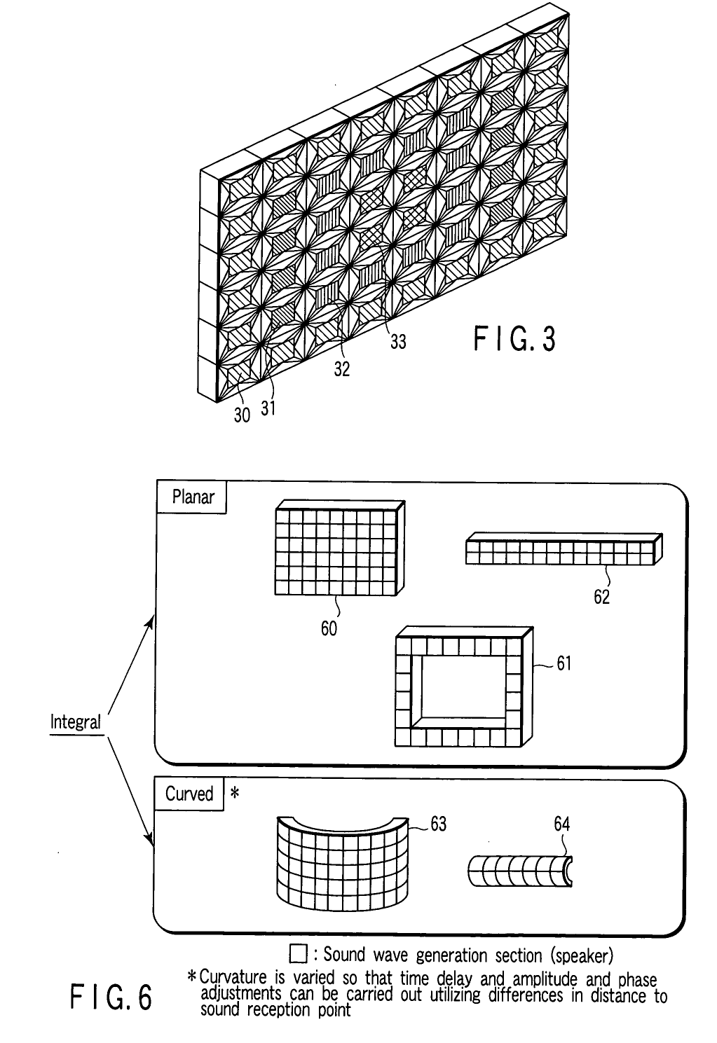

The first embodiment calculates the control filter by noting the number of sound wave generation sections 4 and 6 that suppresses (preferably minimizes) signals from the N sound pressure detection points 11 and M sound pressure detection points 19 as shown in FIG. 1. A precondition for the present embodiment is that a plurality of sounding sections having the same emission characteristic and the same size are present as an integral structure as shown in FIG. 3. Under this condition, a specific description will be given of steps required to calculate a control filter and a required system configuration.

FIG. 3 shows an integral speaker in which a control sound source group is divided into three. A first control sound source group 30 is placed around the outer periphery of the integral speaker like a frame. A second control sound source 31 comprises two columns of line sound sources arranged to sandwich a third control sound source group 32 (and a main sound source group 33) inside the first control sound source group 30. The third control sound source group 32 is placed inside the first control sound source group 30 so as to surround the main sound source group 33 like a frame. The speaker in FIG. 3 has an integral structure including an arbitrary number of speaker elements each of an arbitrary size and an arbitrary shape which are combined together in a matrix so as to have an arbitrary general size and an arbitrary general shape. A control sound source group and a main sound source group are appropriately selected as described above.

For example, three control sound source groups such as those described above are assumed. If these control sound source groups have complex amplitudes qₛ₁, qₛ₂, and qₛ₃, then for evaluation points N₁ and N₂ in the audible area and synthetic sound pressures P_{N1} and P_{N2}, Equation (3) can be given. Here, Fsinj denotes a space transfer function for the space from the i-th control sound source to the j-th evaluation point.
P_{N}_{₁}=Fₛ₁ₙ₁·qₛ₁+Fₛ₂ₙ₁·qₛ₂+Fₛ₃ₙ₁·qₛ₃
P_{N}_{₂}=Fₛ₁ₙ₂·qₛ₁+Fₛ₂ₙ₂·qₛ₂+Fₛ₃ₙ₂·qₛ₃ (3)

Equation (3) can be transformed into Equation (4) using a determinant,

$$\begin{matrix}[ \begin{matrix}{P}_{N 1} \\ {P}_{N 2}\end{matrix} ] = [ \begin{matrix}{F}_{s 1 n 1} & {F}_{s 2 n 1} & {F}_{s 3 n 1} \\ {F}_{s 1 n 2} & {F}_{s 2 n 2} & {F}_{s 3 n 2}\end{matrix} ] \cdot [ \begin{matrix}\begin{matrix}{q}_{s 1} \\ {q}_{s 2}\end{matrix} \\ {q}_{s 3}\end{matrix} ] \\ = [ \begin{matrix}{F}_{s 2 n 1} & {F}_{s 3 n 1} \\ {F}_{s 2 n 2} & {F}_{s 3 n 2}\end{matrix} ] \cdot [ \begin{matrix}{q}_{s 2} \\ {q}_{s 3}\end{matrix} ] + [ \begin{matrix}{F}_{s 1 n 1} \\ {F}_{s 1 n 2}\end{matrix} ] \cdot {q}_{s 1}\end{matrix} \qquad ( 4 )$$

On the basis of P_{N1} [symbol] 0 and P_{N2} [symbol] 0, Equation (5) is given.

$$[ \begin{matrix}{q}_{s 2} \\ {q}_{s 3}\end{matrix} ] = - {[ \begin{matrix}{F}_{s 2 n 1} & {F}_{s 3 n 1} \\ {F}_{s 2 n 12} & {F}_{s 3 n 2}\end{matrix} ]}^{- 1} \cdot [ \begin{matrix}{F}_{s 1 n 1} \\ {F}_{s 1 n 2}\end{matrix} ] \cdot {q}_{s 1} \qquad ( 5 )$$

On the other hand, for the non-audible area, one evaluation point is set and the synthetic sound pressure from the main sound source group and control sound source groups is adjusted to zero. Under the proposed condition, the number of main sound source groups is equal to the number of evaluation points and is thus one.

Under this condition, the synthetic sound pressure at the evaluation point is defined as Q. Then, Equation (6) can be given using Equation (5). Here, Zp denotes a space transfer function for the space from the main sound source to the evaluation point. Zsi denotes a space transfer function for the space from the control sound source to the evaluation point.

$$\begin{matrix}Q = {Z}_{p} \cdot {q}_{p} + {Z}_{s 1} \cdot {q}_{s 1} + {Z}_{s 2} \cdot {q}_{s 2} + {Z}_{s 3} \cdot {q}_{s 3} \\ = {Z}_{p} \cdot {q}_{p} + {Z}_{s 1} \cdot {q}_{s 1} + \begin{matrix}[ {Z}_{s 2} & {Z}_{s 3} ] \cdot [ \begin{matrix}{q}_{s 2} \\ {q}_{s 3}\end{matrix} ]\end{matrix} \\ = {Z}_{p} \cdot {q}_{p} + {Z}_{s 1} \cdot {q}_{s 1} + \begin{matrix}[ {Z}_{s 2} & {Z}_{s 3} ] \cdot [ - {[ \begin{matrix}{F}_{s 2 n 1} & {F}_{s 3 n 1} \\ {F}_{s 2 n 2} & {F}_{s 3 n 2}\end{matrix} ]}^{- 1} \cdot [ \begin{matrix}{F}_{s 1 n 1} \\ {F}_{s 1 n 2}\end{matrix} ] \cdot \end{matrix} {q}_{s 1} ] \\ = {Z}_{p} \cdot {q}_{p} + [ {Z}_{s 1} - [ \begin{matrix}{Z}_{s 2} & {Z}_{s 3}\end{matrix} ] \cdot {[ \begin{matrix}{F}_{s 2 n 1} & {F}_{s 3 n 1} \\ {F}_{s 2 n 2} & {F}_{s 3 n 2}\end{matrix} ]}^{- 1} \cdot [ \begin{matrix}{F}_{s 1 n 1} \\ {F}_{s 1 n 2}\end{matrix} ] ] \cdot {q}_{s 1}\end{matrix} \qquad ( 6 )$$

Consequently, when the synthetic sound pressure is minimized, that is, Q [symbol] 0, the complex amplitude of the first control sound source can be expressed as Equation (7) using the complex amplitude qp of the main sound source.

$${q}_{s 1} = - {[ {Z}_{s 1} - {\begin{matrix}[ {Z}_{s 2} & {Z}_{s 3} ] \cdot \end{matrix} [ \begin{matrix}{F}_{s 2 n 1} & {F}_{s 3 n 1} \\ {F}_{s 2 n 2} & {F}_{s 3 n 2}\end{matrix} ]}^{- 1} \cdot [ \begin{matrix}{F}_{s 1 n 1} \\ {F}_{s 1 n 2}\end{matrix} ] ]}^{- 1} \cdot {Z}_{p} \cdot {q}_{p} \qquad ( 7 )$$

Substituting Equation (7) into Equation (5) enables the complex amplitudes of the second and third control sound sources to be also calculated using the complex amplitude qp of the main sound source.

That is, the known amplitude of the main sound source enables the amplitude of each control sound source to be calculated on the basis of Equations (5) and (7).

If the main sound source is noise from machines, accurately measuring all the sounding sites is difficult. Thus, it is also conventionally difficult to identify the emission characteristics (emission area or size, directionality, and the like) of the sounding sites. It is further difficult to measure all the amplitudes and phases of sounds emitted by these sounding sites. Consequently, some of the characteristics are unknown. In contrast, the acoustic signal reproduction apparatus according to the present embodiment handles known acoustic signals. Accordingly, information on a generation timing sound source and its contents is known, so that the amplitude characteristic of the main sound source is known. This allows the complex amplitudes determined by Equations (5) and (7) to be used as they are. Therefore, the control filter required for audible area separation can be calculated by directly determining space transfer functions F and Z required to derive Equations (5) and (7).

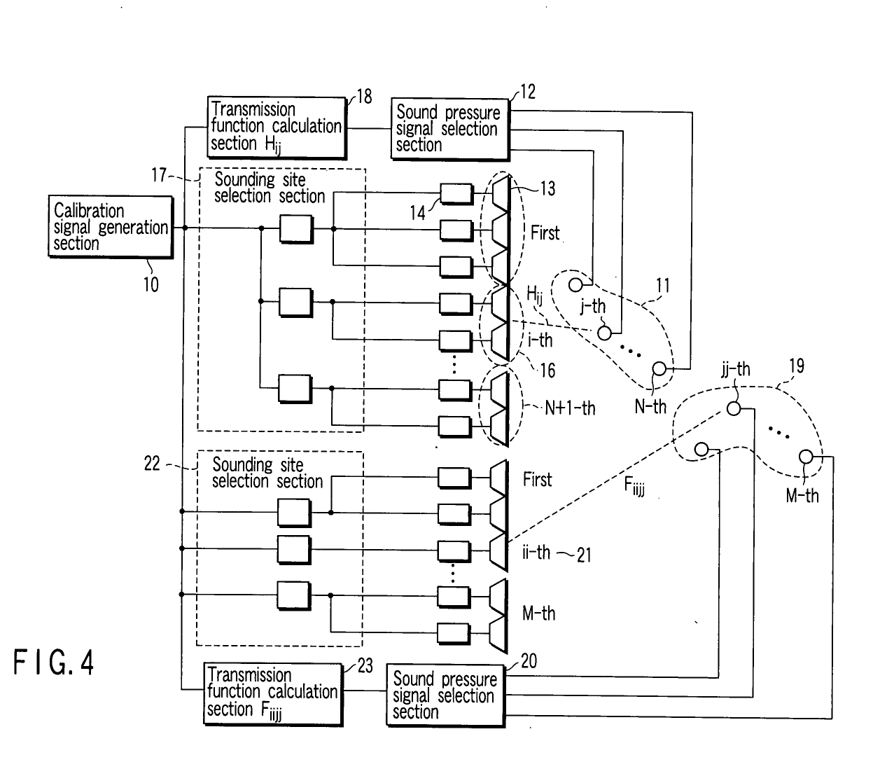

FIG. 4 is a functional block diagram showing means for identifying the space transfer functions F and Z required to derive Equations (5) and (7).

As shown in FIG. 4, this means comprises a calibration signal generation section 10, N sound pressure detection sections 11, a sound pressure signal selection section 12 that selects from sound pressure signals from the N sound pressure detection sections 11, an acoustic wave transmission section comprising a plurality of sound wave generation sections (speakers) 13 and signal amplification sections 14, a sounding site selection section 17 that divides the acoustic wave transmission section into N+1 sites from which sound is selectively generated, and a calculation section 18 that calculates a transfer function on the basis of an acoustic signal provided to the sound wave generation sections 13 by the calibration signal generation section 10 and a sound pressure signal detected by the sound pressure detection section 11. The calculation section 18 sequentially identifies a transfer function Hij (i=1, 2, . . . , N), (j=1, 2, . . . , N+1) for the space from the i-th sounding site 16 to the j-th sound pressure detection section 11.

This means also comprises M sound pressure detection sections 19, a sound pressure signal selection section 20 that selects from sound pressure signals from the M sound pressure detection sections 19, a sounding site selection section 22 that divides a sounding site 21 other than the N+1 divided sounding sites 16 into M portions to allow sound to be selectively generated from the resulting sites, and a calculation section 23 that that calculates a transfer function on the basis of the acoustic signal provided to the sound wave generation sections 13 by the calibration signal generation section 10 and a sound pressure signal detected by the sound pressure detection section 19. The calculation section 23 sequentially identifies a transfer function Fiijj for the space from the i-th sounding site 16 and the ii-th sounding site 21 to the jj-th sound pressure detection section 19.

### Third Embodiment

In a third embodiment, description will be given of calculation (control filter calculation means) of a control filter that achieves the audile area separation described in the first embodiment on the basis of the space transfer function identified by the transfer function identification means described in the second embodiment.

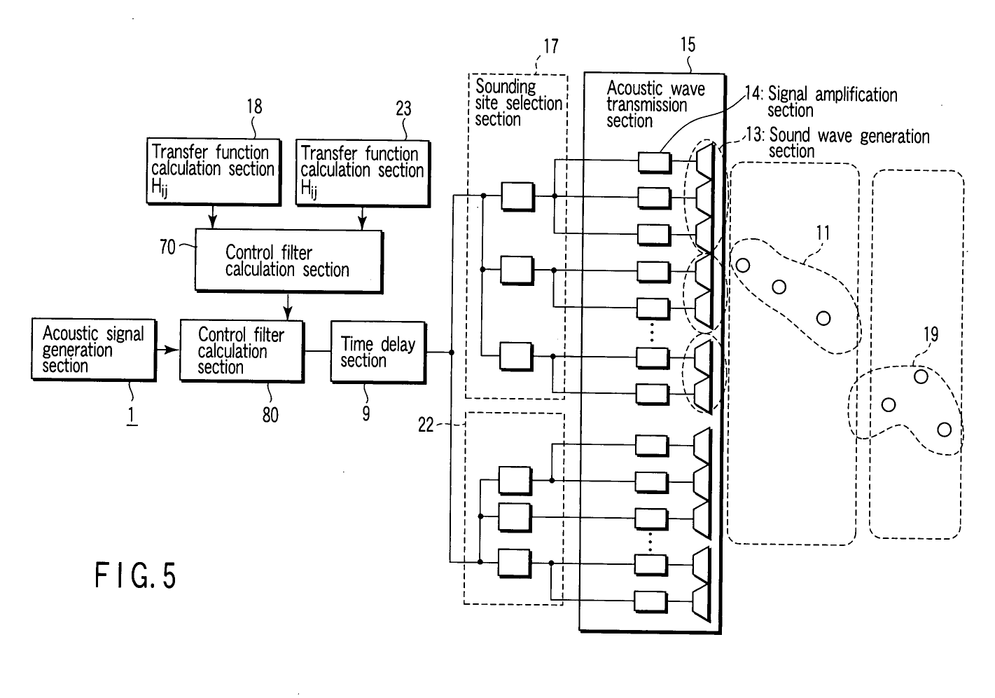

FIG. 5 is a functional block diagram showing control filter calculation means.

As shown in FIG. 5, a control filter calculation section 70 calculates a control filter from the transfer function Hij identified by the transfer function calculation section 18 and the transfer function Fiijj identified by the transfer function calculation section 23. The control filter calculation section 70 provides the calculated control filter to a control filter calculation section 80.

With the configuration shown in FIG. 1, the control calculation section 80 calculates the amplitude and phase of each of the plural control sound sources such that the sum of sound pressure signals from the N+1 control sound wave generation sections 4 is suppressed, preferably minimized, at each of the N sound pressure detection points 3 and such that the sum of sound pressure signals from the N+1 control sound wave generation sections 4 and sound pressure signals from the M sound pressure generation sections 6 is suppressed, preferably minimized, at each of the M sound pressure detection points 5 (step 3 in FIG. 2 (during reproduction of a contents sound and execution of audible sound separation).

Equations (3) and (4), shown in the second embodiment, are intended for calculations for frequency areas. These equations enable the amplitude and phase of the control sound source to be calculated but have no specifications for time areas for which control timings are taken into account. Thus, time delays are required to make the control sound source in time for the generation timings of the main sound source. Accordingly, the time delay sections 9 are provided. Depending on the characteristics of the sound sources, the time delay sections 9 synchronizes the main sound source with the control sound source to allow these sound sources to interfere spatially with each other.

### Fourth Embodiment

A fourth embodiment relates to an example of configuration of a sound wave generation section (speaker).

The N+1 control sound wave generation section 4 and M sound wave generation sections 6, shown in FIG. 1, and the plural sound wave generation sections 13, shown in FIG. 4, are roughly classified into those in which the sound wave generation surface is planar and those in which the sound wave generation surface is curved. The sound wave generation sections with planar wave generation surfaces include a rectangular sound wave generation section 60, a frame like sound wave generation section 61, and a linear (rod-like) sound wave generation section 62. The sound wave generation sections with curved sound wave generation surfaces include sound wave generation sections 63 and 64 shaped like partly cutaway cylinders. The sound wave generation sections with curved sound wave generation surfaces can have their time delays, amplitudes, and phases adjusted by varying their curvatures, utilizing differences in distance to a sound reception point.

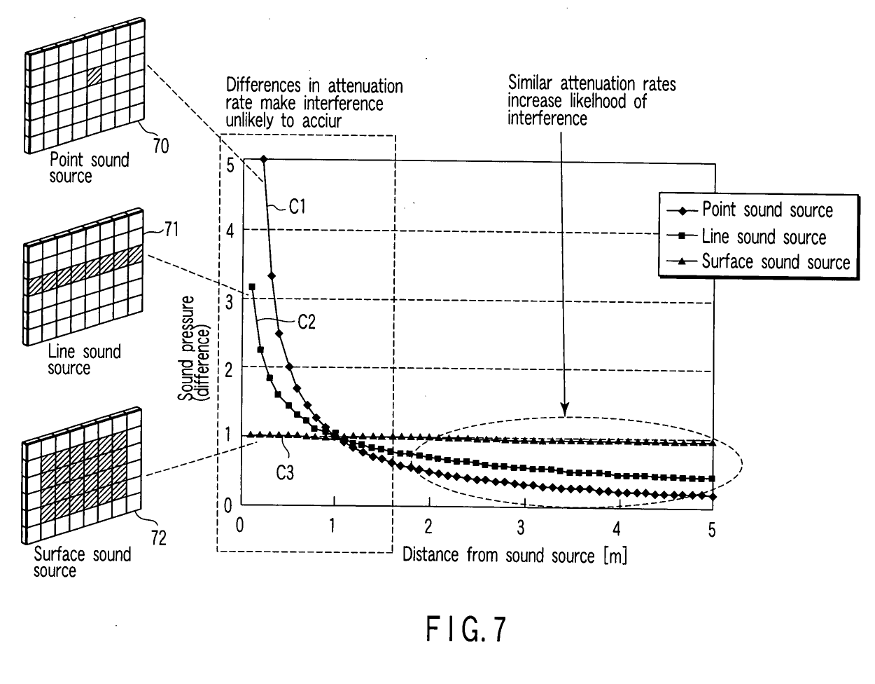

As shown in FIG. 7, sound range attenuation is characterized by varying significantly depending on the size and shape of the emission surface of the sound surface in the vicinity of the sound source, while remaining almost fixed without depending on the size or shape in a remote area. FIG. 7 is a graph showing the relationship between the distance [m] from the sound source and the sound pressure (difference). A curve C1 indicates the point sound source 70. A curve C2 indicates the line sound source 71. A curve C3 indicates the surface sound source 72. As shown in FIG. 7, in a remote area, the sound sources exhibit similar attenuation factors, which increase the likelihood of spatial interference of sound waves. For example, the point sound source 70 is used as a main sound source to reproduce contents. In this case, the line sound source 71 and surface sound source 72 are used as control sound sources.

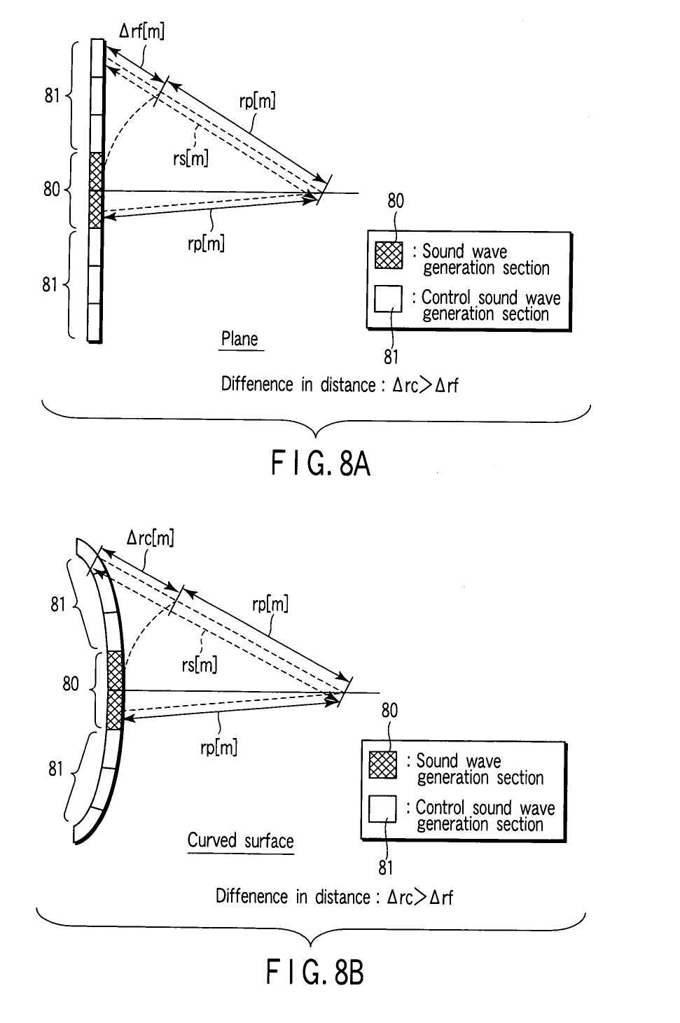

Even the sound wave generation sections with the same surface shape exhibit different range differences depending on the size of the surface and the position of the sound reception point. This also makes these sound wave generation sections exhibit different time delays, amplitudes, and phases. Further, sound wave generation sections with curved surfaces exhibit greater range differences than those with planar surfaces and thus larger time delays, amplitudes, and phase differences. This is shown in FIGS. 8A and 8B. FIG. 8A shows that the sound wave generation surface is planar. FIG. 8B shows that the sound wave generation surface is curved. In FIG. 8A, the sound wave generation section 80 is integrated with control sound generation sections 81 so as to constitute a plane. In FIG. 8B, the sound wave generation section 80 is integrated with control sound generation sections 81 so as to constitute a curved surface. A comparison of FIG. 8A with FIG. 8B indicates that range difference Δr_{c}>range difference Δr_{f}. Thus, different initial phases reduce possible interference around the nearby N sound pressure detection points 3.

By combining this principle with the principle described in the first embodiment to the third embodiment, it is possible to form an audible area (non sound reduction area) around the nearby N sound pressure detection points 3, while forming a non-audible area (sound reduction area) around the remote M sound pressure detection points 5; sound attenuates rapidly in the non-audible area. This makes it possible to achieve sound field area separation.

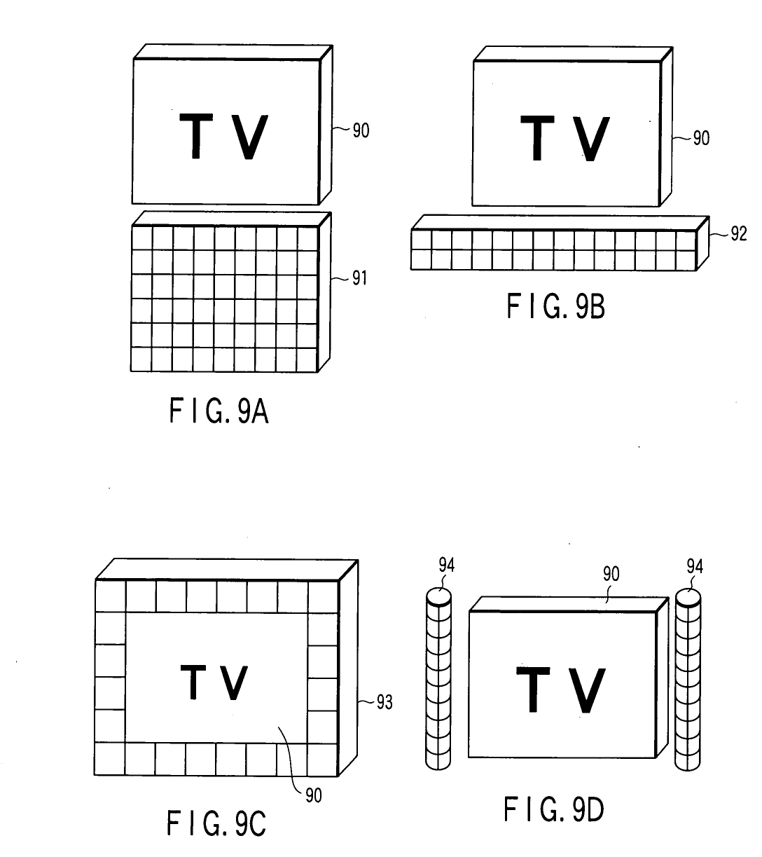

FIGS. 9A and 9B are model drawings showing the present invention is applied to a television. FIG. 9A shows an example of a configuration in which a sound source 91 is placed below the lower end of a television 90. FIG. 9B shows an example of a configuration in which a sound source 92 is placed below the lower end of the television 90 so as to save space. FIG. 9C shows an example of a configuration in which a frame-shaped sound source 93 is placed so as to surround the television 90. FIG. 9D shows an example of a configuration in which cylindrical sound sources 94 are arranged on the opposite sides of the television.

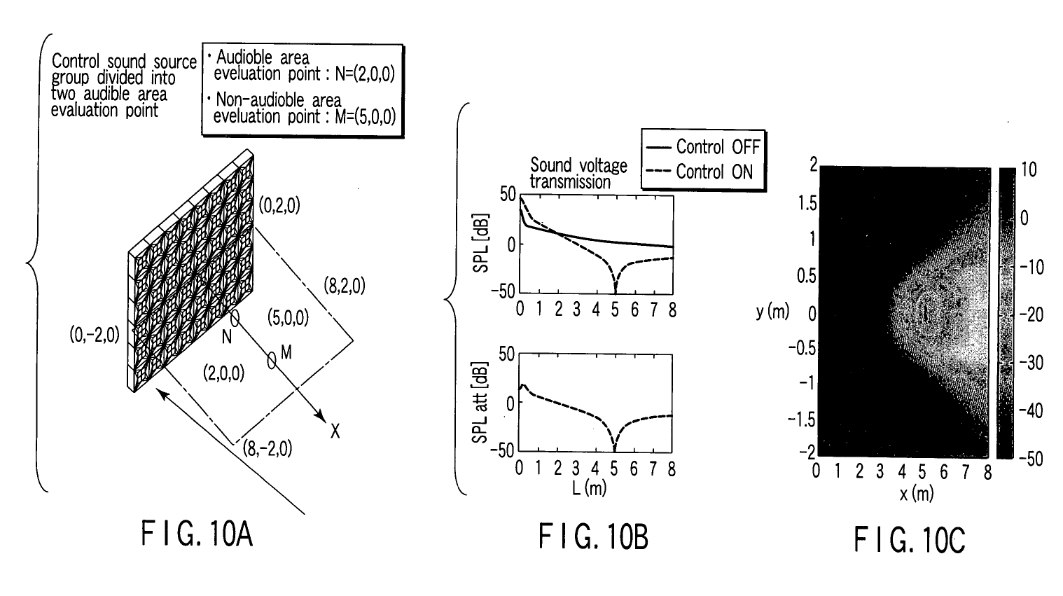

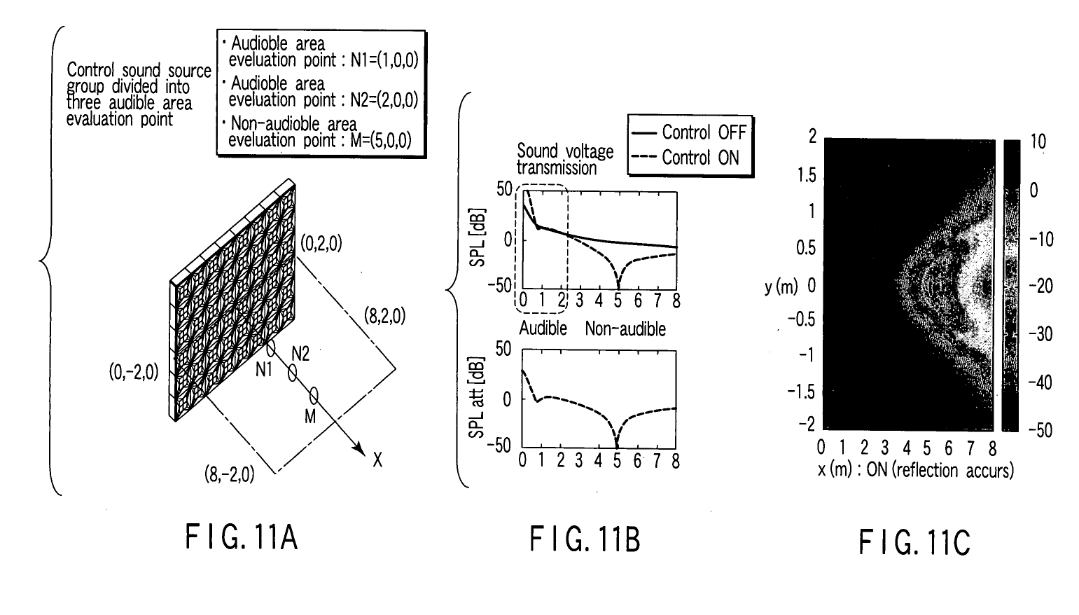

FIGS. 10 and 11 are diagrams showing the results of control performed if the control sound wave generation sections (control sound source group) 4 of the 6×8 integral speaker (FIG. 3) are divided into two or three, respectively.

FIG. 10A shows a model in which the control sound source group is divided into two. FIG. 10B shows the amount of decrease in sound pressure. FIG. 10C shows a sound pressure distribution diagram. In this model, point sound source groups comprising four speakers are provided in a central part of the integral speaker. The control sound source group is divided into a first control sound source group located sp as to surround the outer periphery of the four point sound source groups and a second control sound source group located on the outermost periphery of the integral speaker.

FIG. 11 shows that the control sound source group is divided into three. In this model, point sound source groups comprising four speakers are provided in a central part of the integral speaker. The control sound source group is divided into a first control sound source group located sp as to surround the outer periphery of the four point sound source groups, a second control sound source group located on the outermost periphery of the integral speaker, and a third control sound source group located in the gap between the first control sound source group and the third control sound source group.

As seen in FIGS. 10 and 11, the sound pressure transition and distribution of a control field varies depending on the number of control sound source groups into which the original control sound source group is divided as well as the selected area. A comparison of FIG. 10 with FIG. 11 indicates that the sound pressure drop in the audible area is smaller in FIG. 11 (three control sound source groups). This is a preferable characteristic.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An acoustic signal reproduction apparatus comprising:
an acoustic signal generator which generates an acoustic signal;
first sound pressure detection points located at N (N is a natural number) points in an audible area to detect sound pressure signals;
N+1 control sound wave generators each of which emits a sound wave based on the acoustic signal to generate a control sound;
second sound pressure detection points located at M (M is a natural number) points in a non-audible area to detect sound pressure signals;
M sound wave generators each of which emits a sound wave based on the acoustic signal to generate a main sound; and
a controller configured to control an amplitude and a phase of each of the (N+1) control sound wave generators so as to suppress a sum of first sound pressure signals based on control sounds generated by the N+1 control sound wave generators, the first sound pressure signals being detected by the N first sound pressure detection points, and to suppress a sum of second sound pressure signals from the N+1 sound wave generators and second sound pressure signals from the M sound wave generators, the second pressure signals being detected by the M sound pressure detection points.

2. The apparatus according to claim 1, further comprising a time delay unit which provides a delay time for the control sound in order to synchronize the control sound with the main sound.

3. The apparatus according to claim 1, wherein the main sound and the control sound exhibit different range attenuation factors.

4. The apparatus according to claim 1, further comprising:
a calibration signal generator which generates a calibration signal; and
a space transfer function identification device configured to identify a first space transfer function for a space from each of the N+1 control sound wave generators to each of the N first sound pressure detection points on the basis of the calibration signal,
the space transfer function identification device identifying a second space transfer function for a space from each of the N+1 control sound wave generators and each of the M sound wave generators to each of the M second sound pressure detection points on the basis of the calibration signal.

5. The apparatus according to claim 4, further comprising:
a control filter calculator configured to calculate a control filter used to control, by a filter process, the amplitude and phase of each of the N+1 control sound wave generators on the basis of the first space transfer function and the second space transfer function.

6. The apparatus according to claim 1, wherein a sound wave generation surface formed by the N+1 control sound wave generators is planar.

7. The apparatus according to claim 1, wherein a sound wave generation surface formed by the N+1 control sound wave generators is curved.

8. The apparatus according to claim 1, wherein a sound wave generation surface formed by the M sound wave generators is planar.

9. The apparatus according to claim 1, wherein a sound wave generation surface formed by the M sound wave generators is curved.

10. The apparatus according to claim 1, wherein the N+1 control sound wave generators and the M sound wave generators are arranged in proximity to one another to constitute an integral structure.

11. An acoustic signal reproduction method comprising:
generating an acoustic signal by an acoustic signal generator;
generating a main sound based on the acoustic signal by each of M sound wave generators;
generating a control sound based on the acoustic signal by each of N+1 control sound wave generators;
detecting sound pressure signals by first sound pressure detection points located at N (N is a natural number) points in an audible area;
detecting sound pressure signals by second sound pressure detection points located at M (M is a natural number) points in a non-audible area; and
controlling an amplitude and a phase of each of the (N+1) control sound wave generators so as to suppress a sum of first sound pressure signals based on control sounds generated by the N+1 control sound wave generators, the first sound pressure signals being detected by the N first sound pressure detection points, and to suppress a sum of second sound pressure signals from the N+1 sound wave generators and second sound pressure signals from the M sound wave generators, the second pressure signals being detected by the M sound pressure detection points.

12. The method according to claim 11, further comprising providing a delay time for the control sound in order to synchronize the control sound with the main sound.

13. The method according to claim 11, wherein the main sound and the control sound exhibit different range attenuation factors.

14. The method according to claim 11, further comprising:
identifying a first space transfer function for a space from each of the N+1 control sound wave generators to each of the N first sound pressure detection points on the basis of a calibration signal; and
identifying a second space transfer function for a space from each of the N+1 control sound wave generators and each of the M sound wave generators to each of the M second sound pressure detection points on the basis of the calibration signal.

15. The method according to claim 14, further comprising:
calculating a control filter used to control, by a filter process, the amplitude and phase of each of the N+1 control sound wave generators on the basis of the first space transfer function and the second space transfer function.

16. The method according to claim 11, wherein a sound wave generation surface formed by the N+1 control sound wave generators is planar.

17. The method according to claim 11, wherein a sound wave generation surface formed by the N+1 control sound wave generators is curved.

18. The method according to claim 11, wherein a sound wave generation surface formed by the M sound wave generators is planar.

19. The method according to claim 11, wherein a sound wave generation surface formed by the M sound wave generators is curved.

20. The method according to claim 11, wherein the N+1 control sound wave generators and the M sound wave generators are arranged in proximity to one another to constitute an integral structure.
